(19) United States
(12) Patent Application Publication
(10) Pub. No.: US 2021/0101024 A1
(43) Pub. Date: Apr. 8, 2021

(54) COULOMB-STIMULATION DEVICE FOR BREAKING UP IRON-OXIDE MINERALIZATION WITH PROTEIN AGGREATION USING TRAVERSING ION BEAM AND TRAVERSING ION BEAM DOSIMETER

(71) Applicant: DAEGU CATHOLIC UNIVERSITY INDUSTRY ACADEMIC COOPERATION FOUNDATION, Gyeongsan-si (KR)

(72) Inventor: Jong Ki KIM, Daegu (KR)

(21) Appl. No.: 17/038,905

(22) Filed: Sep. 30, 2020

(30) Foreign Application Priority Data
Oct. 7, 2019 (KR) 10-2019-0124092

(51) Int. Cl. A61N 5/10; G01T 1/02

(52) U.S. Cl. CPC A61N 5/1071; A61N 5/1028; A61N 2005/1076; G01T 1/02; A61N 2005/1087; A61N 5/1077

(57) ABSTRACT

Provided is a headset dosimeter for using a coulomb-stimulation device for breaking up iron-oxide mineralization with protein aggregation using a traversing ion beam comprising a head mounting part, a first stereoscopic block provided on one side to the head mounting part, and a second stereoscopic block provided at an opposite position to the first stereoscopic block based on the head mounting part, wherein each of the first stereoscopic block and the second stereoscopic block may include a mounting part capable of receiving at least one radiographic film.

### BACKGROUND OF THE INVENTION

### Field of the Invention

The present invention relates to an apparatus useful for removing lesions of degenerative brain diseases such as Alzheimer's dementia and Parkin's disease, and a dosimeter for using the same.

### Description of the Related Art

Conventional drug-based therapy technology for Alzheimer's dementia has used a method of limiting a deposition pathway of extracellular amyloid plaques by inhibiting the production of an Aβ protein or enhancing cerebral excretion of the corresponding protein. In addition, there is sought a method of removing an intracellular metal ion pool itself using a chemical metal iron chelator or inactivating the toxicity of a soluble redox-toxic Aβ oligomer.

However, there was limited in the restriction of the passage of a brain blood barrier (BBB) of a drug required to trigger this reaction, removal of redox-toxicity by non-reaction with an iron chelator targeting an intracellular iron pool and redox-toxic iron oxide nanoparticles in pre-produced extracellular insoluble amyloid plaques, and removal of aggregation of amyloid plaques and tau. That is, in the related art, the presence of iron oxide nanoparticle minerals, which are a source of the redox-toxicity, is not known, and thus, target setting of the therapy was incorrect.

In the case of a method using sonication, there is an effect of removing amyloid plaques by releasing the aggregation of the plaques, but due to the temporary releasing phenomenon, there is a high possibility that redox-toxic metal ion-iron oxide nanoparticles still form a conjugate, and after a certain period of time elapses, the protein aggregation is reproduced in the beginning and should be repeated periodically at a 2-week interval, and the like. In addition, since there is no reaction mechanism with the redox-toxic iron oxide nanoparticles, the metal-protein biomineralization in a pathological ferritin protein or in a core of plaque or tau aggregation was not fundamentally removed equally.

The above-described technical configuration is a background technique for assisting the understanding of the present invention, and does not mean a conventional technology widely known in the art to which the present invention belongs.

### SUMMARY OF THE INVENTION

The present invention provides a coulomb-stimulation device capable of detoxicating redox-toxic iron oxide nanoparticles by breaking up or cutting a bond between protein-iron oxide nanoparticles, and removing protein aggregation lesions by irreversibly damaging the bound protein at the same time.

The present invention provides a coulomb-stimulation device capable of breaking up or pulverizing a bond between protein-iron oxide nanoparticles using a traversing ion beam.

The present invention provides a coulomb-stimulation device capable of irreversibly breaking up iron oxide nanoparticles in iron-oxide mineralization with protein aggregation of degenerative brain diseases accompanied with iron-oxide mineralization with protein aggregation lesions, such as β-amyloid plaques, tau aggregation, Lewis body, etc.

The present invention provides an in vitro contacted headset dosimeter capable of accurately measuring a dose of an ion beam, such as cerebral incident and permeable protons, for an accurate procedure using a traversing ion beam.

According to an exemplary embodiment of the present invention, a headset dosimeter for using a coulomb-stimulation device for breaking up iron-oxide mineralization with protein aggregation using a traversing ion beam may comprise a head mounting part, a first stereoscopic block provided on one side to the head mounting part, and a second stereoscopic block provided at an opposite position to the first stereoscopic block based on the head mounting part, wherein each of the first stereoscopic block and the second stereoscopic block m ay include a mounting part capable of receiving at least one radiographic film.

The first stereoscopic block and the second stereoscopic block may be formed using an acrylate or soft tissue-simulant gel.

The head mounting part may include an elastic band, and the mounting part may include film slits formed in the first stereoscopic block and the second stereoscopic block.

A stereotactic position sensor for specifying a position and an angle of the installed stereoscopic block is installed in each of the first stereoscopic block and the second stereoscopic block, and the stereotactic position sensor may generate or receive information on the position and the angle of the stereoscopic block.

According to an exemplary embodiment of the present invention, a coulomb-stimulation device for breaking up iron-oxide mineralization with protein aggregation using a traversing ion beam may comprise a traversing ion beam projector causing coulomb collision by projecting a proton beam of 40 to 300 MeV onto iron oxide nanoparticles spread over the lesions, wherein the traversing ion beam projector may break up or pulverize a bond between the iron oxide nanoparticles-protein as the effect of the coulomb collision, and convert protein aggregation with the iron oxide nanoparticles to be soluble.

The coulomb-stimulation device may further include a headset dosimeter, wherein the headset dosimeter may include a head mounting part, a first stereoscopic block provided on one side to the head mounting part, and a second stereoscopic block provided at an opposite position to the first stereoscopic block based on the head mounting part.

The coulomb-stimulation device for breaking up iron-oxide mineralization with protein aggregation may further include a controller and a plurality of measuring sensors connected to the controller. Correspondingly, a stereotactic position sensor for specifying the position and the angle of the installed stereoscopic block may be installed in each of the first stereoscopic block and the second stereoscopic block in the headset dosimeter.

The stereotactic position sensor may communicate information on the position and the angle of the first and second stereoscopic blocks with the measuring sensors connected to the controller.

By using the coulomb-stimulation device for breaking up the iron-oxide mineralization with protein aggregation using the traversing ion beam of the present invention, it is possible to provide a significant effect of restoring cognitive functions and removing behavioral disorders by inactivating redox-toxicity of degenerative brain diseases such as Alzheimer's dementia, Parkinson's disease, Huntington's disease, ALS, which are accompanied with protein-iron oxide biomineralization induced by iron oxide and pulverizing and dissolving insoluble protein-iron oxide mineralization.

Further, when therapy is performed to patients with early dementia and Parkinson's disease, it is possible to expect an effect of blocking the progress of iron oxide mineralization and prevent the development of clinical symptoms such as dementia by removing an iron oxide storage protein in an abnormal functional-state.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a TEM image showing a state before proton stimulation treatment (top) and a state after treatment (bottom) by using a coulomb-stimulation device for breaking up iron-oxide mineralization with protein aggregation using a traversing ion beam according to an embodiment of the present invention;

FIG. 2 is an STXM-XAS spectrum for detecting iron ion states of magnetite before and after proton stimulation treatment by using the coulomb-stimulation device for breaking up iron-oxide mineralization with protein aggregation using the traversing ion beam according to an embodiment of the present invention;

FIG. 3 is a photograph capable of confirming that amyloid plaques are reduced over time in the cerebral cortex in an APP/PS1 Alzheimer's dementia mouse model after proton stimulation treatment by using the coulomb-stimulation device for breaking up iron-oxide mineralization with protein aggregation using the traversing ion beam according to an embodiment of the present invention;

FIG. 4 is a graph capable of confirming that amyloid plaques are reduced over time in the cerebral cortex in the APP/PS1 Alzheimer's dementia mouse model after proton stimulation treatment as the result of FIG. 3;

FIG. 5 is a graph capable of confirming that toxic magnetite in amyloid plaques is reduced over time in the cerebral cortex in the APP/PS1 Alzheimer's dementia mouse model after proton stimulation treatment as the result of FIG. 3;

FIG. 6 is a perspective view of a headset dosimeter for using the coulomb-stimulation device for breaking up iron-oxide mineralization with protein aggregation using the traversing ion beam according to an embodiment of the present invention;

FIG. 7 is a plan view of the headset dosimeter of FIG. 6;

FIG. 8 is a perspective view for describing a process of mounting the headset dosimeter on a head or a phantom according to an embodiment of the present invention;

FIG. 9 is a plan view for describing a process of using the headset dosimeter mounted on the phantom according to an embodiment of the present invention;

FIG. 10 is a plan view for describing the headset dosimeter according to an embodiment of the present invention; and

FIG. 11 is a plan view for describing the coulomb-stimulation device for breaking up iron-oxide mineralization with protein aggregation using the traversing ion beam and the headset dosimeter according to an embodiment of the present invention.

### DETAILED DESCRIPTION OF THE PREFERRED EMBODIMENT

Hereinafter, preferred embodiments of the present invention will be described in detail with reference to the accompanying drawings, but the present invention is not limited or restricted to the embodiments. For reference, in the description, like reference numerals refer to substantially like components, which may be described by citing contents disclosed in other drawings under such a rule and contents determined to be apparent to those skilled in the art or repeated may be omitted.

A representative lesion of degenerative brain diseases is protein aggregation such as amyloid, tau, and alpha-synuclein, which are covalently bonded with iron oxide nanoparticles as a nucleus, and the protein aggregation has redox-toxicity of iron (Fe2+) and may be a non-soluble complex in which metal ions such as copper and zinc, and the like are additionally aggregated or agglomerated on the surface of the complex.

From this, soluble amyloid oligomers spread in all directions, and may function as a neurotoxic material that damages the surrounding nervous tissues, and the like by redox-toxicity of metal ions and iron oxide.

When a high-energy ion beam is traversing to these lesions, ionization caused by coulomb collision with iron oxide nanoparticles (magnetite or perihydrat) inside a protein-aggregated biomineral and a coulomb nanoradiator effect of emitting low energy electrons such as Auger electrons and interatomic decay electrons (Interatomic Coulomb Decay, ICD) and specific fluorescence X-rays (Fe—K, L line X-rays) occur to break the bond between iron oxide nanoparticles-protein, damage the protein, and convert protein aggregation with the aggregated iron oxide nanoparticles to be soluble. That is, toxic iron oxide (Fe2+ ions) is deactivated (Fe3+ ions), and damaged protein fragments may be removed by the phagocytosis of microglia.

### EXAMPLE 1

An amyloid protein-magnetite fibrin model was prepared in an in vitro test tube in which neurons were cultured, it was confirmed that magnetite was reduced to divalent ions, which were in a redox-toxic state, and it was confirmed through a TEM image and STXM-XAS X-ray microscopy that by permeating 100 MeV protons (1-4 Gy), magnetite was detoxified to trivalent ions with little cell damage, and magnetite-amyloid fibrin was destroyed.

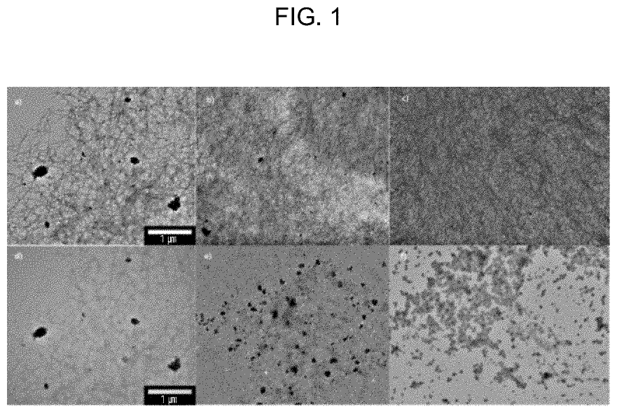

FIG. 1 is a TEM image showing a state before proton stimulation treatment (top) and a state after treatment (bottom) by using a coulomb-stimulation device for breaking up iron-oxide mineralization with protein aggregation using a traversing ion beam according to an embodiment of the present invention.

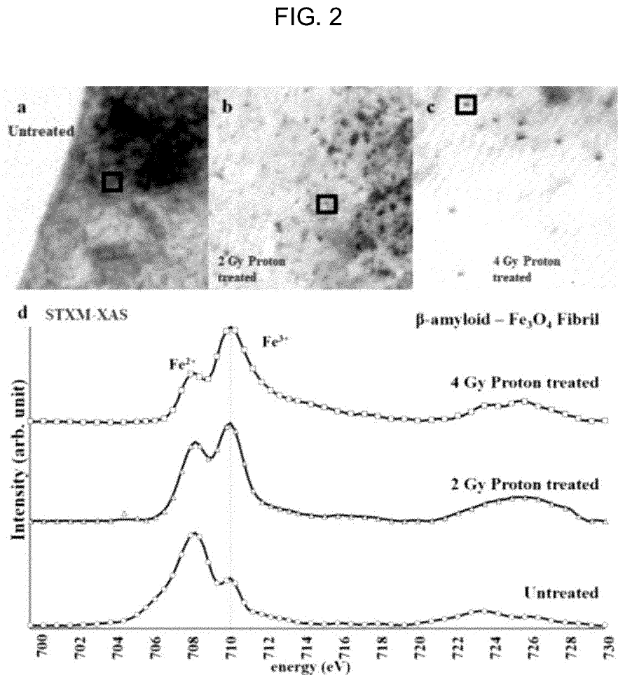

FIG. 2 is an STXM-XAS spectrum for detecting iron ion states of magnetite before and after proton stimulation treatment by using the coulomb-stimulation device for breaking up iron-oxide mineralization with protein aggregation using the traversing ion beam according to an embodiment of the present invention.

Referring to FIG. 2, it can be seen that redox-toxic iron oxide (Fe+2) is converted into non-toxic iron oxide (Fe+3) after treatment.

### EXAMPLE 2

A genetic-variant Alzheimer's dementia mouse model (APP/PS1) test group expressing amyloid plaques was treated by transmitting a 100 MeV proton beam (1-4 Gy), and then sacrificed on day 1, day 3, and day 7, respectively, and hippocampus and cortical tissue specimens were stained with amyloid plaque-congo red and ferrous/ferric iron-Turnbull/Perl's. It was confirmed that amyloid plaques and Fe+2-magnetite were treated with a proton traversing dose and then a therapeutic effect of removing 60% to 95% compared to an untreated control group was obtained in proportion to the passage of the time.

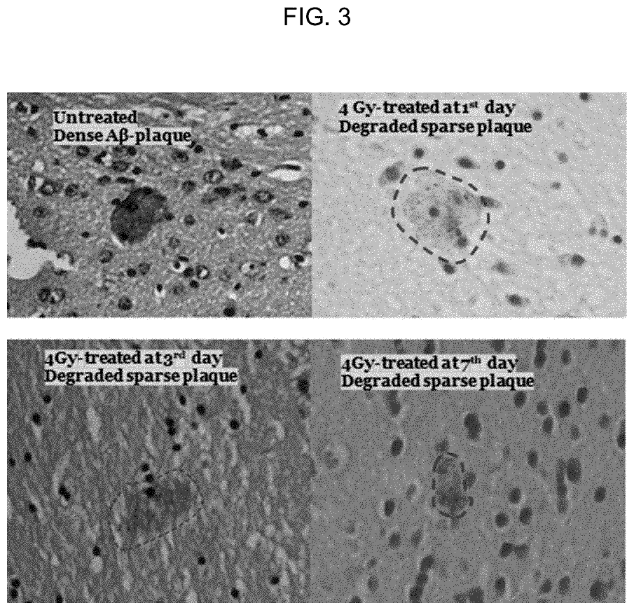

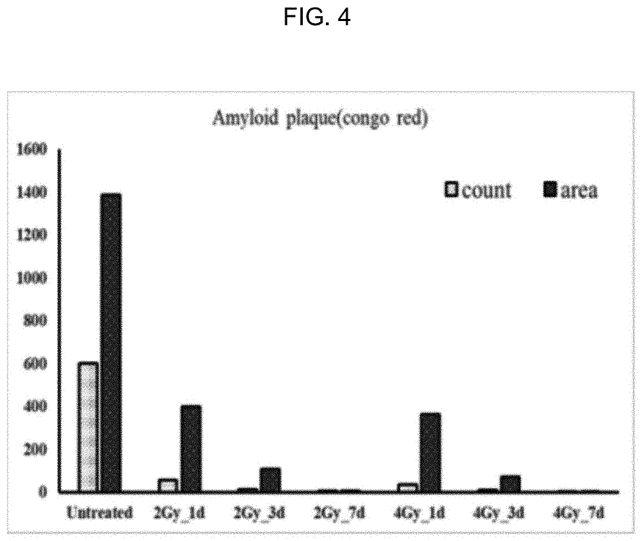

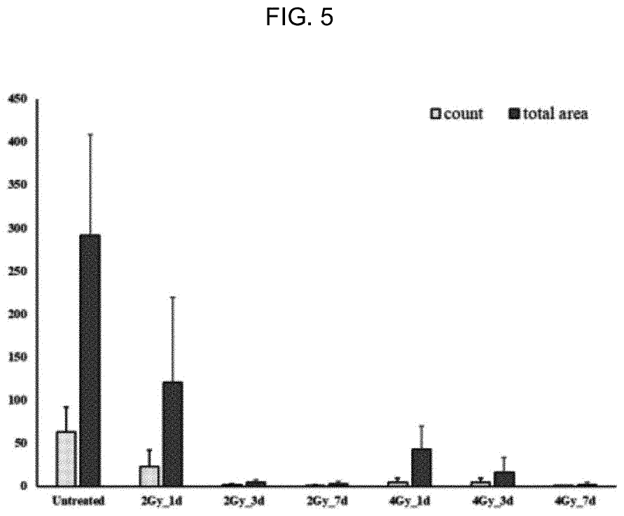

FIG. 3 is a photograph capable of confirming that amyloid plaques are reduced over time in the cerebral cortex in a APP/PS1 Alzheimer's dementia mouse model after proton stimulation treatment by using the coulomb-stimulation device for breaking up iron-oxide mineralization with protein aggregation using the traversing ion beam according to an embodiment of the present invention, FIG. 4 is a graph capable of confirming that amyloid plaques are reduced over time in the cerebral cortex in the APP/PS1 Alzheimer's dementia mouse model after proton stimulation treatment as the result of FIG. 3, and FIG. 5 is a graph capable of confirming that toxic magnetite in amyloid plaques is reduced over time in the cerebral cortex in the APP/PS1 Alzheimer's dementia mouse model after proton stimulation treatment as the result of FIG. 3.

Dosimeter

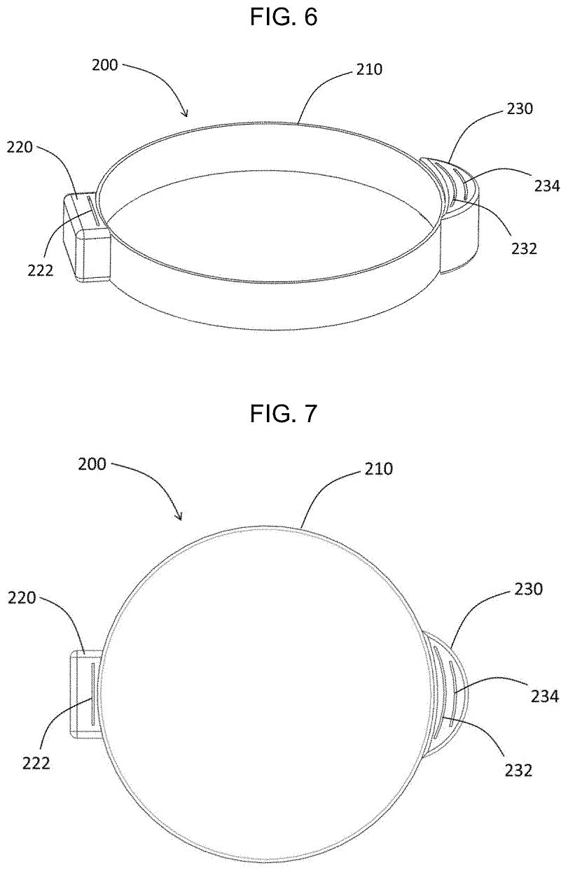

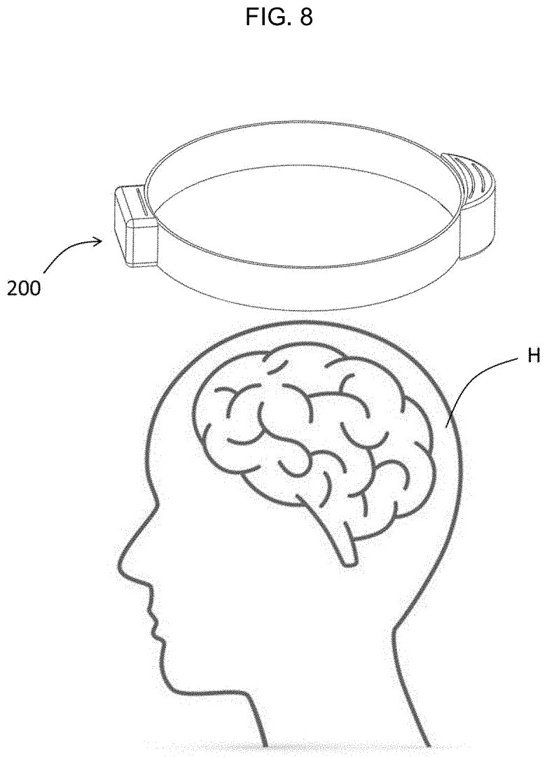

FIG. 6 is a perspective view of a headset dosimeter for using the coulomb-stimulation device for breaking up iron-oxide mineralization with protein aggregation using the traversing ion beam according to an embodiment of the present invention and FIG. 7 is a plan view of the headset dosimeter of FIG. 6.

Referring to FIGS. 6 and 7, a headset dosimeter 200 according to the present embodiment includes a first stereoscopic block 220 and a second stereoscopic block 230 provided in opposite directions with respect to a head mounting part 210, wherein the first stereoscopic block 220 and the second stereoscopic block 230 may be provided as an acrylate or soft tissue-simulant gel. The first stereoscopic block 220 and the second stereoscopic block 230 include a mounting part capable of receiving a radiographic film.

In the embodiment, the mounting part may include a first film slit 222, a second film slit 232, and a third film slit 234, and each film slit may be inserted with the radiographic film or a film-type photosensitive part.

The first stereoscopic block 220 may measure a plateau dose immediately before an incident ion beam enters the cerebrum, and the second stereoscopic block may measure a dose at a point where a Bragg peak dose is formed after passing through the cerebrum.

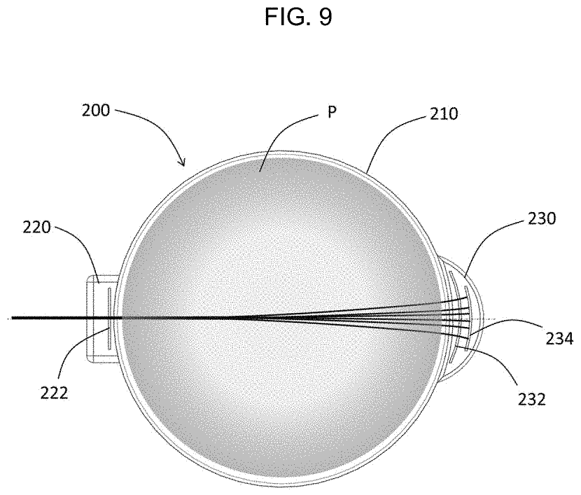

FIG. 8 is a perspective view for describing a process of mounting the headset dosimeter on a head or a phantom according to an embodiment of the present invention and FIG. 9 is a plan view for describing a process of using the headset dosimeter mounted on the phantom according to an embodiment of the present invention.

Referring to FIGS. 8 and 9, the dosimeter 200 may be worn on a patient's head H by using the head mounting part 210 including an elastic band on the patient's head, the incident and traversing doses and a scattering distribution are measured in situ by the worn dosimeter 200, and the ion beam of the traversing dose stimulates and activates the iron oxide nanoparticles to generate coulomb nanoradiator electron emission, thereby attenuating the protein-aggregated lesions.

The headset dosimeter 200 may be pre-mounted with a model phantom P of a patient before treatment, that is, a 3D skull-brain model phantom for cerebral image-based, and may be used for a target-transmission model experiment of a traversing ion beam. Through this, the headset dosimeter 200 may be used for improving the accuracy of target setting by three-dimensionally simulating the Bragg peak dose and the incident amount for each transmission path.

The radiation damage sensitivity may vary depending on a region. By measuring and controlling the dose of the ion beam passing through the cerebral region in advance in the phantom, only the lesion may be treated and removed while preventing damage to a normal tissue during treatment. For example, the hippocampus region may have higher sensitivity to radiation damage than that of the occipital/frontal lobes.

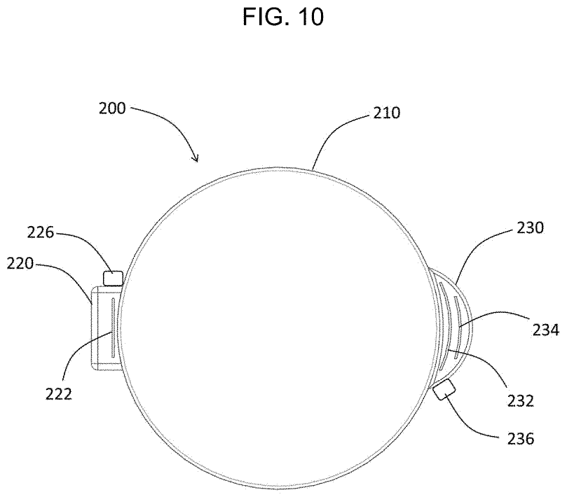

FIG. 10 is a plan view for describing the headset dosimeter according to an embodiment of the present invention.

Referring to FIG. 10, the headset dosimeter 200 may include a head mounting part 210, a first stereoscopic block 220, a second stereoscopic block 230, and the like, and may further include stereotactic position sensors 226 and 236 for equally maintaining the positions and angles of the stereoscopic blocks.

A coulomb-stimulation device capable of generating a traversing ion beam may also utilize a proton computed tomography and may generate or receive a signal capable of specifying positions, and the like on the two stereotactic position sensors 226 and 236. By using the stereotactic position sensors 226 and 236, the coulomb-stimulation device or the headset dosimeter 200 may check whether the current headset dosimeter 200 is disposed in the same manner as the stored position and angle, and may indicate how much displacement or angle the position is distorted if not matched.

This process may assist in performing the same treatment or treatment process as before.

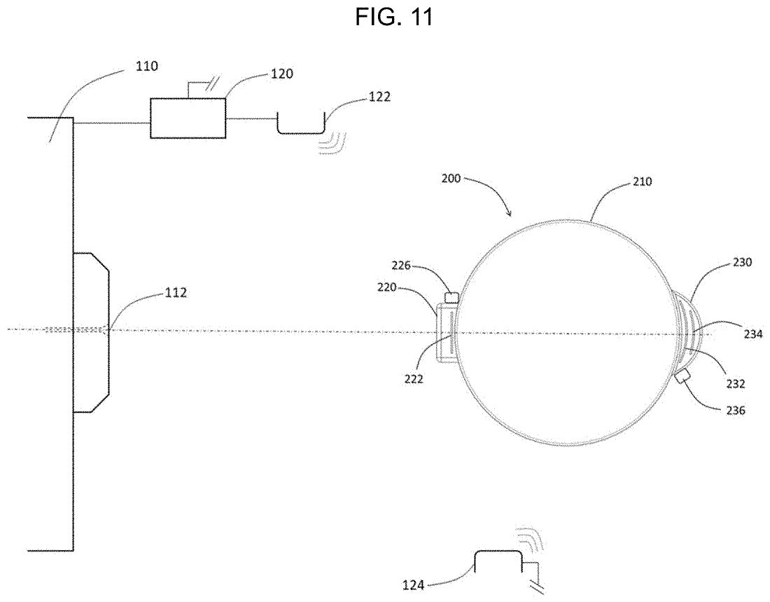

FIG. 11 is a plan view for describing the coulomb-stimulation device for breaking up iron-oxide mineralization with protein aggregation using the traversing ion beam and the headset dosimeter according to an embodiment of the present invention.

Referring to FIG. 11, the coulomb-stimulation device for breaking up iron-oxide mineralization with protein aggregation includes a traversing ion beam projector 110. The traversing ion beam projector 110 generates a proton beam of 40 to 300 MeV, and the proton beam may cause coulomb collisions with iron oxide nanoparticles spread over the lesions.

As an effect of the coulomb collision, the bond between the iron oxide nanoparticles-protein may be broken or pulverized, the corresponding protein may be damaged, and the protein aggregation with the iron oxide nanoparticles distributed and aggregated into hundreds to thousands may be converted to be soluble.

The headset dosimeter measures and controls the dose of the ion beam passing through the cerebral region in advance in the phantom to treat and remove only the lesion while preventing damage to a normal tissue during treatment and increase the sensitivity of radiation damage in a hippocampus region compared to the occipital/frontal lobes.

To this end, the coulomb-stimulation device for breaking up iron-oxide mineralization with protein aggregation may further include a controller 120 and a plurality of sensors 122 and 124 connected to the controller 120. The sensors 122 and 124 of the projector record a position and a posture of the headset dosimeter by the stereotactic position sensors 226 and 236 of the headset dosimeter and may be used for adjusting the position and the posture of the headset dosimeter depending on the recorded values, or for comparing other data before and after. The description of the headset dosimeter may refer to the description and drawings of the previous embodiments.

As described above, the present invention has been described with reference to the preferred embodiments. However, it will be appreciated by those skilled in the art that various modifications and changes of the present invention can be made without departing from the spirit and the scope of the present invention which are disclosed in the appended claims.

What is claimed is:

1. A headset dosimeter for using a coulomb-stimulation device for breaking up iron-oxide mineralization with protein aggregation using a traversing ion beam, the headset dosimeter comprising:
a head mounting part;
a first stereoscopic block provided on one side to the head mounting part; and
a second stereoscopic block provided at an opposite position to the first stereoscopic block based on the head mounting part,
wherein each of the first stereoscopic block and the second stereoscopic block includes a mounting part capable of receiving at least one radiographic film.

2. The headset dosimeter of claim 1, wherein the first stereoscopic block and the second stereoscopic block are formed using an acrylate or soft tissue-simulant gel.

3. The headset dosimeter of claim 1, wherein the head mounting part includes an elastic band, and the mounting part includes film slits formed in the first stereoscopic block and the second stereoscopic block.

4. The headset dosimeter of claim 1, wherein a stereotactic position sensor for specifying a position and an angle of the installed stereoscopic block is installed in each of the first stereoscopic block and the second stereoscopic block, and the stereotactic position sensor generates or receives information on the position and the angle of the stereoscopic block.

5. A coulomb-stimulation device for breaking up iron-oxide mineralization with protein aggregation using a traversing ion beam, the coulomb-stimulation device comprising:
a traversing ion beam projector or a proton tomography causing coulomb collision by projecting a proton beam of 40 to 300 MeV onto iron oxide nanoparticles spread over the lesions,
wherein the traversing ion beam projector breaks up a bond between the iron oxide nanoparticles-protein as the effect of the coulomb collision, and converts protein aggregation with the iron oxide nanoparticles to be soluble.

6. The coulomb-stimulation device of claim 5, further comprising:
a headset dosimeter comprising a head mounting part, a first stereoscopic block provided on one side to the head mounting part, and a second stereoscopic block provided at an opposite position to the first stereoscopic block based on the head mounting part,
wherein each of the first stereoscopic block and the second stereoscopic block includes a mounting part capable of receiving at least one radiographic film.

7. The coulomb-stimulation device of claim 6, wherein the first stereoscopic block and the second stereoscopic block are formed by using an acrylate or soft tissue-simulant gel.

8. The coulomb-stimulation device of claim 6, wherein the head mounting part includes an elastic band, and the mounting part includes film slits formed in the first stereoscopic block and the second stereoscopic block.

9. The coulomb-stimulation device of claim 6, further comprising:
a controller and a plurality of measuring sensors connected to the controller,
wherein a stereotactic position sensor for specifying the position and the angle of the installed stereoscopic block is installed in each of the first stereoscopic block and the second stereoscopic block, and
the stereotactic position sensor communicates information on the position and the angle of the first and second stereoscopic blocks with the measuring sensors connected to the controller.

10. A method for breaking up coulomb-stimulated iron-oxide mineralization with protein aggregation using a traversing ion beam, the method comprising:
causing coulomb collision by projecting a proton beam of 40 to 300 MeV onto iron oxide nanoparticles spread over the lesions, wherein the traversing ion beam breaks up a bond between the iron oxide nanoparticles-protein as the effect of the coulomb collision, and converts protein aggregation with the iron oxide nanoparticles to be soluble.
